(19) United States
(12) Patent Application Publication
(10) Pub. No.: US 2002/0094337 A1
(43) Pub. Date: Jul. 18, 2002

(54) Prevention of recurrent viral disease

(75) Inventors: Laure Aurelian, Baltimore, MD (US); Takahiro Gyotoku, Fukuoko (JP)

Correspondence Address: MORGAN, LEWIS & BOCKIUS LLP, 1701 MARKET STREET, PHILADELPHIA, PA, 19103-2921, US

(73) Assignee: University of Maryland, Baltimore

(21) Appl. No.: 09/992,453

(22) Filed: Nov. 16, 2001

Related U.S. Application Data
(60) Provisional application No. 60/249,387, filed on Nov. 16, 2000.

(51) Int. Cl. A61K 39/245

(52) U.S. Cl. 424/231.1

(57) ABSTRACT

The present invention discloses compositions and methods for ameliorating or reducing recurrent viral disease, which compositions and methods result in an increase in virus specific immunoglobulin subclasses reflective of a preferential Th1 response.

### CROSS REFERENCE TO RELATED APPLICATIONS

The present application claims priority under 35 U.S.C. § 119(e) to U.S. Provisional Application No. 60/249,387 filed on Nov. 16, 2000.

### STATEMENT REGARDING FEDERALLY SUPPORTED RESEARCH OR DEVELOPMENT

This invention was made in part using funds obtained from the U.S. Government (National Institute of Allergy and Infectious Diseases Grant No. 960184). The U.S. Government may have certain rights in this invention.

### BACKGROUND OF THE INVENTION

The spread of sexually transmitted diseases continues unabated, despite educational efforts made in response to the epidemic of human immunodeficiency virus (HIV). Recent studies indicate that the age-adjusted prevalence of herpes simplex virus type 2 (HSV-2) in the US is now 20.8%, an increase of approximately 30% over the past 13 years. Overall it is 50% homologous to type 1 virus (HSV-1), which causes facial lesions. However, the two viruses have a predilection for different body sites, a different propensity to cause recurrent disease (60% and 30% for HSV-2 and HSV-1 respectively), they are associated with different neurological diseases, primarily meningitis for HSV-2 and encephalitis for HSV-1, and only HSV-2 has neoplastic potential. The increasing rate of HSV-2 acquisition among young adults increases the likelihood that infants will be exposed to HSV-2 at delivery, resulting in an infection that, despite antiviral therapy, is still life-threatening. New concerns about HSV-2 infection are that it causes previously undescribed hyperproliferative lesions and it facilitates the spread of HIV as well as increasing the severity of the disease.

Infection with either HSV-1 or HSV-2 can be divided into four stages: (i) acute infection, (ii) establishment of latency, (iii) maintenance of the latent state, and (iv) reactivation of latent virus. The most common site of primary HSV infection is at mucosal membranes, facial for HSV-1 and genital for HSV-2. The virus replicates in cells at the site of infection, resulting in primary lesions. Virus DNA is retained in sensory neurons in a latent state, generally throughout the lifetime of the host. Certain stimuli cause reactivation of virus replication with concomitant reverse axonal transport of virus progeny to a peripheral site, at or near the portal of entry. Periodic reactivation of the latent viral genome results in virus replication often causing recurrent disease. However, reactivation does not always result in recurrent disease. Only a fraction of the infected subjects (60% and 30% for HSV-2 and HSV-1 respectively) develop recurrent disease. HSV infection is followed by the development of humoral and T cell mediated immunity. Infected subjects have relatively high titers of virus specific antibody (IgG and IgM) and T cell responses that persist for their lifetime, and the outcome of infection is affected by the immune status, with immunosuppressed individuals sustaining severe, debilitating disease.

Recurrent HSV-2 lesions are linked to transient downregulation of virus-specific T cell responses, both in the guinea pig model of recurrent disease (Iwasaka et al., 1983, Infect. Immun. 42:955-964) and in infected patients. In human patients, T cell downregulation was first seen during prodrome (1-2 days before lesion onset) at the time of neuronal virus reactivation and was no longer seen on day 3-5 after lesion onset when symptoms begin to clear. Down regulation seemed to reflect a shift in the balance of HSV-specific T helper cells in favor of the type 2 (Th2) population that has down-regulatory function, as evidenced by increased levels of Th2 cytokines, e.g., IL-6 and IL-10 and concomitant decrease in the levels of Th1 cytokines, e.g., interferon gamma (IFN-γ).

Co-administration of Th1 cytokine genes was shown to increase the potency of DNA prophylactic vaccines (Sin et al., 1999, J. Immunol. 162:2912-2921). However, administration of Th1 cytokines or IL-12 is not a promising approach to prevent recurrent disease development, since these factors contribute to the pathogenesis of some HSV diseases, such as keratitis (Niemialtowski et al., 1992, J. Immunol. 149:3035-3039) or HSV-associated erythema multiforme (Jones et al., 2000, J. Gen. Virol. 81:Pt 2:407-414), and their administration in this environment culminates in increased severity of immunopathologic HSV disease (Kanangat et al., 1996, J. Immunol. 156:1110-1116). Furthermore, virus reactivation in latently infected trigeminal ganglia is most effectively inhibited by CD8+ cytotoxic T cells (CTL) (Liu et al., 2000, J. Exp. Med. 191:1459-1466), indicating that administration of Th1 cytokines alone will not prevent recurrent disease. However, CD8+ CTL are induced poorly, if at all, by HSV infection due to interference by a virus protein (ICP47) (Jugovic et al., 1998, J. Virol. 72:5076-84).

Pachuk et al., U.S. Pat. No. 5,958,895, discloses constructs allowing a shift of the immune response from primarily Th1 to primarily Th2 for the design of improved HSV vaccine protocols. Pachuk et al. further states that a Th2 response will afford the vaccinee improved protection. However, no indication of the response desired for reduction of recurrent disease is proposed.

Ghiasi et al. found that CD4+ including CD8+ CTL cells are both involved in protection against HSV-1 (Ghiasi et al., 2000, Br. J. Ophthalmol. 84(4):408-12). Ghiasi, (U.S. Pat. No. 6,193,984), also found that complex mixtures of HSV proteins could be used to generate antibodies at a level below that found in HSV infected animals.

Studies of vaccinia recombinants indicated that: (i) glycosylation-related epitopes are essential for the induction of protective immunity by viral glycoproteins, (ii) protection is achieved by vaccination with non-structural HSV proteins, (iii) development of protective immunity depends on “relevant” antigen presentation which is predicated on the construction of the recombinant vector and (iv) protection from fatal HSV-2 disease and clearance of high dose HSV-2 from the skin are mainly a function of virus-specific T helper type 1 (Th1) immune responses, but CD8+ CTL are also involved (Wachsman et al., 1992, Vaccine 10:447-454). The role of the virus specific immune response in preventing recurrent disease is unclear. Immunization with antigen-encoding plasmid DNA was shown to induce protective immunity in some models but immunity was incomplete in others.

Relatively little is known about therapeutic vaccines. Subunit preparations caused a modest reduction in recurrent disease frequency and severity of symptoms, and reduced the incidence of viral shedding, but these effects were dependent on the co-administration of potent adjuvants (Ho et al., 1989, J. Virol. 63:2951-2958). Minimal reduction (36%) in cumulative lesion score was seen with a gH deleted HSV recombinant given by one (but not another) route (Boursnell et al., 1997, J. Inf. Dis. 175:16-25), and the role of the immune response is still unclear.

ICP10ΔPK is a mutant HSV-2 virus which has a deletion in the protein kinase (PK) domain of the ICP10 gene. This mutant and its properties as a vaccine have been described in U.S. Pat. Nos. 6,013,265, 6,054,131, and 6,207,168. The ICP10ΔPK mutant does not cause neoplastic transformation and fails to activate the mitogenic/proliferative Ras/MEK/MAPK pathway (Aurelian et al., 1999, Vaccine 17:1951-1963; Smith et al., 2000, J.Virol. 74:10417-10429). This feature is clinically significant because recent studies indicate that HSV-2 can cause extensive hyperproliferative lesions in infected patients (Beasley et al., 1997, J. Am. Acad. Dermatol. 37:860-863).

ICP10ΔPK retains a broad antigenic spectrum for presentation to the immune system. It induces HSV-specific humoral and T cell immunity in the mouse model (Aurelian et al. 1999, Vaccine 17:1951-1963) and a DTH response in the guinea pig (Wachsman et al., 2001, Vaccine 19:1879-1890). However, it is becoming increasingly evident that immunization of HSV-2 infected animals can modulate the existing virus specific immune response toward increased levels of Th1 or Th2-like profiles and this modulation is dependent on the form of the antigenic stimulus (Mohamedi et al., 2000, Vaccine 18, 1778-1792). Because ICP10ΔPK is shown herein to prevent the development of recurrent disease (i.e., is a therapeutic vaccine), it provides a unique model to guide the selection of other constructs capable of reducing recurrent disease in herpes, in which the virus recurs periodically or is present over long periods of time.

There is a long felt need in the art to provide a mechanism by which useful therapeutic vaccines which ameliorate recurrent disease can be identified and used. The present invention satisfies this need.

### BRIEF SUMMARY OF THE INVENTION

This invention relates to preventing or reducing the symptoms of recurrent herpes simplex virus related disease in an animal latently infected with HSV by inducing a Th1 response in the animal.

### DETAILED DESCRIPTION OF THE INVENTION

The present invention teaches eliciting a particular immune response, namely, a T Helper Cell type 1 (Th1) response. More specifically, the present invention teaches a method of eliciting an increase in a Th1 response as compared to a Th2 response. Furthermore, the present invention teaches compounds, and how to identify such compounds, which elicit an increase in a Th1 response as compared to a Th2 response. Such response typically comprises one or more of the following responses: an increased ratio of virus specific immunoglobulin subclasses reflective of a preferential Th1 response, an increased ratio of IFNγ/IL-10, increased IL-12 levels, and increased CD8+CTL levels, that are specific for a latently infecting HSV, and thereby protecting a latently infected animal from recurrence of disease symptoms associated with reactivation of HSV. The increased ratio of virus specific immunoglobulin subclasses reflective of a preferential Th1 response in mice is an increased IgG2a/IgG1 ratio as described herein. However, in humans and other animals it is possible that the immunoglobulins of the response and their ratios may vary and includes such ratios as IgG1/IgG4, IgG2/IgG4, IgG3/IgG4, (IgG1+IgG2+IgG3)/IgG4, (IgG1+IgG2+IgG3)/IgG5, IgG1/IgE, IgG2/IgE, or IgG3/IgE.

In one particular embodiment, the present invention teaches that live HSV-2 lacking the ICP10PK domain, and hence the associated protein kinase and oncogene activities, induces a unique immune response, that this unique immune response persists in a subject infected with live wild type HSV-2, and that this unique immune response can be used in an assay to identify other HSVs or mutant HSVs, compounds, compositions, agents or molecules that can also induce this protective unique immune response. Therefore, a novel method has been discovered for eliciting a specific immune response associated with immunity against HSV infection and recurrent disease as well as a novel method of identifying agents which induce this immune response.

In one embodiment of the invention, HSV, or mixtures of proteins from HSV lacking the ICP6PK or ICP10PK protein, is administered with or without immune stimulants or adjuvants to induce a predominant viral specific Th1 response. Such proteins can also be administered indirectly by administering HSV DNA, or mixtures of DNA or nucleic acids that encode HSV proteins lacking the DNA that encodes ICP6PK or ICP10PK protein, with or without immune stimulants or adjuvants to induce a predominant virus specific Th1 response. In either case, the virus specific Th1 response comprises an increase in the ratio of virus specific immunoglobulin subclasses reflective of a preferential Th1 response such as IgG2a/IgG1 in mice or IgG1/IgG4, IgG2/IgG4, IgG3/IgG4, (IgG1+IgG2+IgG3)/IgG4, (IgG1+IgG2+IgG3)/IgG5, IgG1/IgE, IgG2/IgE, or IgG3/IgE in humans, over the pre-administration ratio by at least 15%, preferably by at least 25%, thus providing a method to reduce recurrent disease. In an even more preferred embodiment, the increase in the ratio is by at least 50%. In a more preferred embodiment, the increase in the ratio is by at least 75%. In the most preferred embodiment, the increase in the ratio is by at least 95%. Based on the disclosure provided herein, one of skill in the art will know that Th1 responses may vary among different species, both in immunoglobulin subclasses and ratios, and will be able to use the appropriate techniques to determine and measure the response.

Additionally or instead of one or more of the increased immunoglobulin ratios, the increased CD8+ CTL levels, and the increased IL-12 levels, the virus specific Th1 response can comprise an increase in the viral-specific IFNγ/IL-10 ratio over the pre-administration ratio by at least 15%, preferably by at least 25%, as measured either by in vitro culture of T cells or as blood levels, thus providing a method to reduce recurrent disease. In an even more preferred embodiment, the increase in the ratio is by at least 50%. In a more preferred embodiment, the increase in the ratio is by at least 75%. In the most preferred embodiment, the increase in the ratio is by at least 95%.

Additionally or instead of one or more of the increased immunoglobulin ratios, the IFNγ/IL-10 ratios, and CD8+ CTL levels, the virus specific Th1 response can comprise an increase in the IL-12 levels over the pre-administration ratio by at least 15%, preferably by at least 25%, as measured either by in vitro culture of dendritic cells or as blood levels, thus providing a method to reduce recurrent disease. In another embodiment, the increase is by at least 50%. In a more preferred embodiment, the increase is by at least 75%. In the most preferred embodiment, the increase is by at least 95%.

Additionally or instead of one or more of the increased immunoglobulin ratios, IFNγ/IL-10 ratios, and the IL-12 levels, the viral specific CD8+ CTL levels are increased by at least 15%, more preferably by at least 25%, over pre-administration levels, thus providing a method to reduce recurrent disease. In another embodiment, the increase is by at least 50%. In a more preferred embodiment, the increase at least 75%. In the most preferred embodiment, the increase is by at least 95%.

The relative increases in the immunoglobulin subclasses reflective of a preferential Th1 response such as IgG2a/IgG1 seen in mice or IgG1/IgG4, IgG2/IgG4, IgG3/IgG4, (IgG1+IgG2+IgG3)/IgG4, (IgG1+IgG2+IgG3)/IgG5, IgG1/IgE, IgG2/IgE, or IgG3/IgE in humans, the IFNγ/IL-10 ratio, the IL-12 levels, and the CD8+ CTL levels need not all be the same, although it is preferable that all are increased to some degree.

ICP10ΔPK is a mutant HSV-2 virus which has a deletion in the protein kinase (PK) domain of the ICP10 gene. This mutant and its properties as a vaccine have been described in U.S. Pat. Nos. 6,013,265, 6,054,131, and 6,207,168. The HSV-2 mutant ICP10ΔPK is one known way of providing a mixture of proteins that elicit these immune properties. U.S. Pat. Nos. 6,013,265, 6,054,131, and 6,207,168 are incorporated by reference as if set forth in their entirety herein.

ICP6PK is the HSV-1 analog of ICP10PK in HSV-2. To treat recurrent infection with HSV-1, proteins from HSV-1, but not ICP6PK, can be used in the same manner as described herein for HSV-2. The invention should be construed to include all mixtures of HSV proteins from all HSV strains in which the ICP6PK or ICP10PK is not present.

In the present invention, it is shown that treatment of mice with a mixture of HSV proteins in which the ICP10PK is not present, leads to different effects on lymph node cell responses to an HSV-2 challenge than does treatment of the animals with HSV-2. Lymph node cells from the treated mice secrete greater levels of IFN-γ and lower levels of IL-10 when subsequently challenged with HSV-2, than lymph node cells from animals pretreated with HSV-2 proteins containing ICP10 PK. The IFN-γ to IL-10 ratio is also higher in cells derived from animals treated with the HSV protein mixture deficient in ICP10 PK. Furthermore, these exhibit serum levels of the types of viral specific IgG antibodies (IgG2a/IgG1 ratio) which indicate a viral specific Th1 response. Additionally they exhibit increased levels of virus specific CD8+ CTL. These data show that pretreatment in vivo with an appropriately chosen mixture of HSV proteins in which the ICP10PK is not present skews the virus specific immune response toward Th1 functions, resulting in reduction of recurrent disease.

The present invention further illustrates that a virus specific Th1 response is induced by pretreatment of animals with an appropriately chosen mixture having HSV proteins in which the ICP6PK or ICP10PK is not present, based on the increased production of IL-12 by dendritic lymph node cells subsequent to HSV infection.

The invention should not be construed such that the term “administering protein or proteins” is restricted to mean only administering a protein directly. It should also be construed to mean indirect administration of a protein, such as when DNA or an isolated nucleic acid encoding the protein is administered.

Immunizing a subject indicates the standard interpretation well known in the art as well as the therapeutic use of compositions and methods of the invention disclosed herein to reduce symptoms of recurrent disease in a subject latently infected with a pathogenic virus.

The invention also comprises a method of identifying or screening for agents that induce a Th1 immune response against latently infecting pathogens and a method for administering such compositions to animals, preferably humans, to induce such response and thereby to protect the animals against recurrent disease associated with other pathogens. Typically, such other compositions would be prepared by preparing mutant virus strains or protein mixtures, based on the disclosure provided herein.

The formulation of agents that induce a virus specific Th1 immune response for human use is accomplished by suspension in a solution with or without stabilizing ingredients, and with or without immune stimulants and adjuvants. Examples of stabilizing agents, immune stimulants and adjuvants include among others, alum, oil/water emulsions, saponins, incomplete Freund's adjuvants, MR-59 (Chiron Corp., Emeryville, Calif.), MTPPE, and MPL (mono-phosphoryl Lipid A). Such stabilizing agents, adjuvants and immune stimulants are well known in the art and can be used singly or in combination. Stimulants that accentuate production of IgG2 in humans or IgG2a in mice are especially preferred.

The compositions of the present invention can be administered to any animal, including fish, amphibians, birds, and mammals (where mammals include, but are not limited to, monkeys, pigs, horses, cows, dogs, cats, and humans). The compositions may be administered via any suitable mode of administration, such as intramuscular, oral, subcutaneous, intradermal, intravaginal, rectal, or intranasal administration. The preferred modes of administration are oral, intravenous, subcutaneous, intramuscular or intradermal administration. The most preferred mode is parenteral, including subcutaneous administration.

The frequency of administration, including boosters if required, and other techniques associated with immunization are well known to those skilled in the art and if not already described or determined can be done so without undue experimentation. For example, the appropriate immunoprotective, non-toxic, and unique immune response-inducing amount of the composition of this invention may be in the range of the effective amounts of antigen in conventional vaccines. It will be understood however, that the specific dose level for any particular subject will depend upon a variety of factors including the age, general health, sex, and diet of the subject. Other factors influencing dose level include, but are not limited to, the time of administration, the route of administration, synergistic, additive, or antagonistic interactions with any other drugs being administered, and the amount of protection or the level of induction of the immune response being sought. For example, in a combination vaccine, the dosage of the vaccine of the present invention may need to be increased to offset the interference of the other vaccine components.

The compositions of the present invention, e.g., a therapeutic vaccine comprising the HSV-2 mutant, ICP10ΔPK, can be used in combination with other HSV vaccines using methods well known to those skilled in the art. Various regimens of exposure to HSV and subsequent administration of vaccines or combination vaccines are included and can be determined using methods well known to those skilled in the art, based on the disclosure provided herein. For example, following exposure of a subject to HSV or a mutant HSV, a subject could be administered various combinations of an HSV vaccine and other HSV vaccines, including HSV-1, HSV-2, mutants of HSV-1, and mutants of HSV-2. The various combinations can be determined by those skilled in the art, based on the disclosure provided herein.

Mutant HSV viruses or other agents that induce a virus specific Th1 immune response, can be administered along with a pharmaceutically acceptable carrier or diluent. Examples of such pharmaceutically acceptable carriers or diluents include water, phosphate buffered saline or sodium bicarbonate buffer. A number of other acceptable carriers or diluents are also known in the art.

Therefore, the present invention has discovered a novel or unique immune response against HSV, a novel method for inducing the response, a novel method for identifying agents which induce the response, and a novel method for ameliorating recurrent viral disease.

Definitions

The articles “a” and “an” are used herein to refer to one or to more than one (i.e. to at least one) of the grammatical object of the article. By way of example, “an element” means one element or more than one element. “Plurality” means at least two.

As used herein the term “about” or “at least” is used to mean approximately 5 percentage points lesser or greater than the number described, i.e., “at least 25% ” means “at least 20 to 30%”, but does not restrict any ranges defined by the phrase “at least”.

As used herein, the term “ameliorate” refers to a treatment which improves or lessens the symptoms of an infection or disease and which prevents or lessens the development of symptoms associated with the active stages of a recurrent disease. Amelioration encompasses both reducing the severity of recurrence as well as the incidence of recurrence of disease. “Ameliorating recurrent disease” is used interchangeably with “reducing recurrent disease”.

By the term “co-administering,” as used herein, is meant before, simultaneously, or subsequently.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins (such as IL-1α, IL-1β, IL-2, IL-3, IL-4, IL-5, IL-6, IL-7, IL-9 (P40), IL-10, IL-11, IL-12 and IL-13); CSF-type cytokines (such as GM-CSF, G-CSF, M-CSF, LIF, EPO, TNF-α and TNF-β); interferons (such as IFN-α, IFN-β and IFN-γ); cytokines of the TGF-β family (such as TGF-β1, TGF-β2, TGF-β3, inhibin A, inhibin B, activin A, and activin B); chemotactic factors (such as NAP-1, MCP-1, MIP-1α, MIP-1β, MIP-2, SISβ, SISδ, SISε, PF-4, PBP, γIP-10, and MGSA); growth factors (such as EGF, TGF-α, aFGF, bFGF, KGF, PDGF-A, PDGF-B, PD-ECGF, INS, IGF-I, IGF-II, and NGF-β); α-type intercrine cytokines (such as IL-8, GRO/MGSA, PF-4, PBP/CTAP/βTG, IP-10, MIP-2, KC, and 9E3); and β-type intercrine cytokines (such as MCAF, ACT-2/PAT, 744/G26, LD-78/PAT 464, RANTES, G26, 1309, JE, TCA3, MIP-1α,β, and CRG-2). A number of other cytokines are also known to those of skill in the art. The sources, characteristics, targets and effector activities of these cytokines have been described.

A “disease”, as used herein, is a state of health of an animal wherein the animal cannot maintain homeostasis.

By the term “herpes” is meant a disease associated with herpes simplex virus.

By the term “immunizing” against an antigen is meant administering to the subject a composition, a protein complex, a DNA encoding a protein complex, an antibody or a DNA encoding an antibody, a phage containing DNA which encodes for a protein or an antibody, or a phage which expresses a protein or antibody on its surface, which elicits an immune response in the subject. Preferably the immune response provides protection to the subject against a disease caused by the antigen or an organism which expresses the antigen.

An “isolated nucleic acid” as used herein refers to a nucleic acid segment or fragment which has been separated from sequences which flank it in a naturally occurring state. The term also refers to nucleic acids which have been substantially purified from other components which naturally accompany the nucleic acid, e.g., RNA or DNA or proteins, which naturally accompany it in the cell.

It is not intended that the present invention be limited by the nature of the nucleic acid employed. The target nucleic acid may be native or synthesized nucleic acid. The nucleic acid may be from a viral, bacterial, animal, phage, or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89.

Nucleic acids useful in the present invention include, by way of example and not limitation, oligonucleotides and polynucleotides; DNA for gene therapy; viral fragments including viral DNA and/or RNA; DNA and/or RNA chimeras; mRNA; plasmids; cosmids; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well known in the art. RNAs may be produced in high yield via in vitro transcription using plasmids.

In some circumstances, as where increased nuclease stability is desired, nucleic acids having modified intemucleoside linkages may be preferred. Nucleic acids containing modified internucleoside linkages may also be synthesized using reagents and methods that are well known in the art. For example, methods for synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH₂—S—CH₂), dimethylene-sulfoxide (CH₂—SO—CH₂), dimethylene-sulfone (—CH₂—SO₂—CH₂), 2′-O-alkyl, and 2′-deoxy-2′-fluoro phosphorothioate internucleoside linkages are well known in the art (see Uhlmann et al., 1990, Chem. Rev. 90:543-584; Schneider et al., 1990, Tetrahedron Lett. 3 1:335 and references cited therein).

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified. The term nucleic acid also specifically includes nucleic acids composed of bases other than the five biologically occurring bases (adenine, guanine, thymine, cytosine and uracil).

As used herein, the term “pharmaceutically acceptable carrier” means a chemical composition with which the active ingredient may be combined and which, following the combination, can be used to administer the active ingredient to a subject.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides or post-transcriptionally altered polypeptides.

The term “peptide” typically refers to short polypeptides or post-transcriptionally altered polypeptides.

By the term “recurrent disease,” as used herein, is meant disease symptoms that occur or re-occur following reactivation of a latent virus.

“Therapeutic,” as used herein, refers to a treatment, administered to a subject who has the disease or exhibits signs of the disease, which is sufficient to provide a beneficial effect. A beneficial effect includes such things as reducing recurrent disease. A prophylactic or preventive treatment or vaccine is one administered to a subject who does not have the disease or exhibits signs of the disease for the purpose of preventing infection and decreasing the risk of developing pathology associated with the disease.

By the term “vaccine,” as used herein, is meant a composition which when inoculated into an animal has the effect of stimulating an immune response in the animal which serves to fully or partially protect the animal against a disease or its symptoms. The term vaccine encompasses prophylactic as well as therapeutic vaccines. A combination vaccine is one which combines two or more vaccines.

### EXAMPLES

The examples presented below are provided as a further guide to the practitioner of ordinary skill in the art and to facilitate a further understanding of the present invention, and are not to be construed as limiting the invention in any way. The examples are illustrative and not limiting of the invention.

The materials used in examples 1-4 presented herein are now described.

Five week old Swiss Webster or BALB/c mice were used and were obtained from Charles River Labs, Wilmington, Mass. Mice were chosen because they are the standard animal model for HSV-2. Anti-IFN-γ antibody (R4-6A2), anti-IL-10 antibody (JESS-2A5), biotinylated anti-IFN-γ antibody (XMG1.2), anti-IL-12 antibody (C15.6), biotinylated anti-IL-12 antibody (C17.8) and anti-IL-10 antibody (JES5-16E3), were obtained from Pharmingen, San Diego, Calif. Goat anti-mouse IgG, IgG1, and IgG2a and mouse anti-human IgG1 and IgG2 were obtained from Southern Biotechnology Associates, Inc, Birmingham, Ala. Antibody coated Dynabeads were obtained from Dynal, Oslo, Norway. Recombinant murine IL-12 was obtained from Pharmingen. Nitrocellulose membranes (Millititer HA) were obtained from Millipore, Bedford, Mass. HSV-2 antigen was obtained from Southern Biotechnology Associates, Inc., Birmingham, Ala.

### Example 1

HSV specific immune response elicited by ICP10ΔPK evidences a predominantly Th1 pattern

The methods used in the experiments presented in this Example are now described.

Mice (Swiss Webster, 5 weeks old, Charles River) were infected with HSV-2 (1×10⁶ plaque forming units (pfu)) or ICP10ΔPK (3×10⁶ pfu) by subcutaneous inoculation in the footpad. They were given 2 or 3 injections at 10 days intervals. Popliteal lymph node cells (LNC) were collected at 3, 5 and 10 days after the last injection and cultured (4×10⁶ cells/ml) in RPMI-10% FCS (complete medium) with 10 μg/ml of HSV-2 antigen. The cells which secrete interferon-gamma (IFN-γ) (Th1) or interleukin 10 (IL-10) (Th2) were identified in ELISPOT assays on days 1-5 in culture. Freshly isolated LNC were also studied for IFN-γ and IL-10 secreting cells using the ELISPOT assay. Also, in some experiments, the LNC were depleted of CD4+ or CD8+ T cells using antibody coated magnetic beads (Dynabeads; Dynal, Oslo, Norway), cultured with HSV-2 antigen (10 μg/ml) and assayed by ELISPOT as above.

ELISPOT assays were performed as previously described. Briefly, 96-well plates containing a nitrocellulose membrane base (Millititer HA, Millipore) were coated with anti-IFN-γ) mAb (R4-6A2: Pharmingen, San Diego, Calif.) or anti-IL-10 mAb (JES5-2A5: Pharmingen) at a concentration of 2 μg/ml (100 μl/well) in PBS, at 4° C. overnight. Wells were washed with PBS twice and then blocked with complete medium for 1 hour at room temperature. Stimulated or unstimulated LNC were added to individual wells (1×10⁵ cells/well) and incubated for 20 hours at 37° C. Wells were washed with PBS-0.5% Tween20 (4×) to remove the cells and incubated for 1 hour at room temperature with 100 μl of biotinylated anti-IFN-γ mAb (XMG1.2: Pharmingen) or anti-IL-10 mAb (JES5-16E3: Pharmingen) at a concentration of 2 μg/ml in PBS-Tween. After washing with PBS-Tween, the reaction was developed by incubation (30 minutes; room temperature) with 100 μl of avidin-peroxidase diluted 1:1000 in PBS-Tween followed by 3-amino-9-ethylcarbazole (AEC). Spots representing colonies of cells that secrete specific cytokines (SFC) were counted and the results from three independent experiments were averaged. The results are expressed as SFC/10⁵ cells +/− SEM.

In LNC from mice given 2 immunizations, the numbers of cells secreting IFN-γ peaked on day 3 post-inoculation for both HSV-2 and ICP10ΔPK and decreased thereafter. At 3 days post-inoculation the SFC/10⁵ cells were 138 +/−20 for HSV-2 and 182 +/−40 for ICP10ΔPK. However, the number of cells secreting IL-10 was higher for HSV-2 than ICP10ΔPK at both 3 and 5 days post-infection, such that the ratios of IFN-γ/IL-10 SFCs were significantly higher for ICP10ΔPK (3.8+/−0.38 and 4.0+/−0.36 on days 3 and 5 post-infection respectively) than HSV-2 (2.0+/−0.13 and 2 +/−0.07 on days 3 and 5 post-infection respectively). A higher ratio of IFN-γ/IL-10 SFCs was still seen for ICP10ΔPK at 10 days post-infection (4.4 +/−1.1 and 2.1 +/−0.08 for ICP10ΔPK and HSV-2 respectively). The IFN-γ/IL-10 ratio was also higher in freshly isolated LNC from mice infected with ICP10ΔPK (5.3 +/−0.39) as compared to HSV-2 (3.4 +/−0.16). The number of cells secreting IFN-γ or IL-10 increased with time in culture, both for LNC from HSV-2 and ICP10ΔPK infected mice, reaching maximal levels on days 2-3 and decreasing thereafter. On day 5 in culture, IL-10 producing cells were still seen for HSV-2 but not ICP10ΔPK. This difference is reflected in a higher IFN-γ/IL-10 SFC ratio for ICP10ΔPK than HSV-2 at this time. In animals given 3 immunizations the IFN-γ/IL-10 SFC ratio of LNC collected at 10 days post-infection was also higher for ICP10ΔPK (5.3 +/−0.7) than HSV-2 (1.9 +/−0.2). The data can be interpreted to indicate that in vivo exposure to ICP10ΔPK skewed the response in favor of Th1 functions and in vitro re-exposure to HSV-2 antigen did not negatively affect this balance.

To confirm the validity of this conclusion, supernatants of the LNC cultures studied above were assayed for the levels of IFN-γ and IL-10. ELISA was performed using anti-IFN-γ (R4-6A2: Pharmingen) and anti-IL-10 (JESS-2A5: Pharmingen) mAbs as capture antibodies and biotinylated anti-IFN-γ (XMG1.2:Pharmingen) mAb and anti-IL-10 (JES5-16E3:Pharmingen) mAb as detection antibodies. Recombinant mouse IFN-γ and IL-10 (Pharmingen) were used as controls to generate standard curves. The results of these studies indicated that the levels of IL-10 were significantly lower in supernatants of HSV-stimulated (or unstimulated) LNC from ICP10ΔPK than HSV-2 immunized animals (924 +/−102 and 4184 +/−358 pg/ml respectively). In both the ELISPOT and ELISA assays, depletion studies indicated that both IFN-γ and IL-10 are produced by CD4+ cells. Thus, the number of cytokine secreting cell colonies in LNC from HSV-2 infected mice (77 +/−5.6 and 37.2 +/−4.2 for IFN-γ and IL-10 respectively) were significantly reduced by depletion of CD4+ cells (7.6 +/−1.3 and 3.8 +/−1.0 for IFN-γ and IL-10 respectively), but depletion of CD8+ CTL cells did not decrease the number of cytokine secreting cells (70 +/−11 and 35.5 +/−5.6 for IFN-γ and IL-10 respectively). Similar results were obtained for the levels of cytokines in the supernatants. In cultures of LNC from HSV-2 infected mice, IFN-γ levels were 7834 +/−578 and 7608 +/−513 pg/ml for unfractionated and CD8-depleted cultures, but only 677+/−51 pg/ml for CD4-depleted cultures. IFN-γ levels in cultures of LNC from ICP10ΔPK infected mice were 4822 +/−191, 4724 +/−493 and 512 +/−36 pg/ml for unfractionated, CD 8-depleted and CD4-depleted cultures respectively. IL-10 levels were 4184 +/−358, 3847 +/−265 and 378 +/−3.5 pg/ml for unfractionated, CD8-depleted and CD4-depleted LNC cultures from HSV-2 infected animals and 924 +/−103, 869 +/−63 and 78 +/−3 pg/ml for unfractionated, CD8-depleted and CD4-depleted LNC cultures from ICP10ΔPK infected animals. Because animals were given two injections and LNC were collected at 1-10 days after the last infection, secondary exposure to ICP10ΔPK appears to enhance the local HSV-specific Th1 response while similar infection with HSV-2 favors the HSV-specific Th2 response.

The conclusion that ICP10ΔPK favors Th1 functions is also supported by the type of IgG antibody that is induced by the two viruses. Mice were infected in the footpad with HSV-2 (10⁶ pfu) or ICP10ΔPK (3×10⁶ pfu) 3 times at 10 day intervals. Sera were collected at 14 days after the last infection and assayed for HSV-specific IgG, IgG1 (dependent on Th2 cells) and IgG2a (dependent on Th2 cells) by ELISA. Briefly, ELISA plates were coated with HSV-2 antigen (50 μg/ml) or goat anti-mouse IgG (Southern Biotechnology Associates Inc., Birmingham, Ala.) (2 μg/ml) used as control for standard curves, and incubated overnight at 4° C. The plates were washed with PBS-Tween and blocked with PBS-10% FCS for 1 hour at 37° C. For the standard curves, serial dilutions of IgG, IgG1 or IgG2a (Southern Biotechnology Associates) were added to the control plates while serial dilutions of the serum samples were added to the HSV coated plates. After 2 hours at 37° C., the wells were washed and goat anti-mouse IgG, IgG1 or IgG2a heavy chain specific antibody conjugated to horseradish peroxidase (Southern Biotechnology Associates) was added. After incubation for 1 hour at 37° C. and subsequent washing, the substrate 2,2-azino-bis 3-ethylbenz-thiasoline-6-sulfonic acid (ABTS) was added to the wells for color development. The wells were read at 405 nm with an ELISA 2550 EIA reader (BioRad, Hercules, Calif.). Total levels of HSV-specific IgG were higher for ICP10ΔPK (7581 +/−2052 ng/ml) than for HSV-2 (3676 +/−735 ng/ml). The levels of HSV-specific IgG1 were higher for ICP10ΔPK than HSV-2 (1609 +/−408 and 381 +/−60 ng/ml respectively) as were those of IgG2a (3338 +/−774 and 537 +/−99 ng/ml for ICP10ΔPK and HSV-2 respectively). The balance of IgG isotypes expressed as the IgG2a/IgG1 ratio was in favor of IgG2a for ICP10ΔPK (2.1 +/−0.17), but not HSV-2 (1.41 +/−0.19).

### Example 2

ICP10ΔPK immunization causes high levels of IL-12 production by dendritic cells

Dendritic cells are involved in determining whether an immune response is Th1 or Th2 based on the levels of IL-12 that they produce in response to antigen. High IL-12 levels skew the response in favor of Th1, including IFN-γ production (Riffaubt et al., 2000, J. Gen. Virol. 8 1:2365-2373).

The materials and methods used in the experiments presented in this Example are now described.

This series of experiments was designed to examine whether IL-12 production by dendritic cells from ICP10ΔPK infected animals is also higher than that seen in animals infected with HSV-2. BALB/c mice (5 weeks old, Charles River) were given 3 injections with HSV-2 (10⁶ pfU) or ICP10ΔPK (3×10⁶ pfu) in the footpad at 10-day intervals and popliteal LNC were collected 24 hours after the last boost. Dendritic cells were isolated by metrizamide gradient centrifugation. Briefly, 2 ml of 14.5% metrizamide (Sigma, Saint Louis, Mo.) in complete medium were layered on the bottom of a 15 ml conical test tube and gently overlaid with 8 ml of the cell suspension. After centrifugation (600× g, 20 minutes, 4° C.), the interface which is enriched in dendritic cells was collected and the cells were cultured (6×10⁴/well) in 96-well round bottom plates with 25 ng/ml of GM-CSF and 5 ng/ml of IL-4 and with or without HSV-2 antigen (10 μg/ml). Culture supernatants were replaced with fresh medium on days 3 and day 5 in culture. At 12 hours after the last medium replacement, culture supernatants were collected and the concentrations of IL-12 were measured by ELISA. The assay was performed as before using anti-IL-12 (C15.6: Pharmingen) mAb as capture antibody, biotinylated anti-IL-12 (C17.8: Pharmingen) mAb as detection antibody and recombinant murine IL-12 (Pharmingen) as control for standard curves. The data indicate that the levels of IL-12 were significantly higher in cultures of dendritic cells from mice given ICP10ΔPK (63.6 +/−8 ng/ml) than those given HSV-2 (17.2 +/−0.6 ng/ml). IL-12 levels were similar in cultures done in the presence or absence of HSV-2 antigen, indicating that increased IL-12 production does not depend upon additional exposure to antigen (in culture).

### Example 3

ICP10ΔPK induces CD8+ CTL

Previous studies have shown that CD8+ CTL activity is enhanced by increased levels of IL-12 and IFN-γ (McNally et al., 1999, Immunology 163:675-681). Having shown that ICP10ΔPK modulates the immune response towards increased IL-12 production by dendritic cells and enhanced Th1 responses (higher levels of IFN-γ), it was next determined whether it also skews the response in favor of CD8+ CTL.

The materials and methods used in the experiments presented in this Example are now described.

BALB/c mice were infected twice at 10-day intervals. Infection was in the footpad with HSV-2 (1×10⁶ pfu) or ICP10ΔPK (3×10⁶ pfu). An HSV-2 mutant deleted in the RR domain of ICP10 (ICP10ΔRR; 3×10⁶ pfu) was used as control for non-specific effects related to infection with mutant viruses. Popliteal LNC were collected 5 days after the last boost. Cells (2×10⁶/ml) were cultured in complete medium for 3 days at 37° C. Nonadherent cells were washed once with complete medium and used as effectors. In some experiments, LNC were depleted of CD4+ or CD8+ CTL cells using antibody coated Dynabeads (Dynal) prior to in vitro culture. mKSA (H-2^{d}) cells grown in Dulbecco's modified medium (DMEM) with 10% heat-inactivated FCS were infected with 10 PFU/cell of HSV-2 for 17 hours and labeled with 100 μCi of ⁵¹Cr for the last 16 hours. They were washed with DMEM, trypsinized, washed three additional times with DMEM and the viable cells were used as targets. They were resuspended in complete medium to a final concentration of 2×10⁵ cells/ml and distributed (2×10⁴ cells/well in 100 ml) into 96-well round-bottom plates containing equal volumes of effectors adjusted to yield the desired effector to target ratio (E:T). The plates were centrifuged (200× g, 2 minutes), incubated at 37° C. for 6 hours, centrifuged again (200× g, 5 minutes), and the ⁵¹Cr released into the supernatant fluid was counted in a Beckman Gamma Counter (Beckman Coulter, Fullerton, Calif.). Spontaneous release was determined by incubating labeled target cells without effector cells. Maximum release was determined by lysing the target cells with 5% SDS. The percent specific cytotoxic activity was determined from the following formula: % specific ⁵¹Cr Release=Experimental Release Spontaneous Release/Maximum Release−Spontaneous Release.

The CTL activity of unfractionated LNC from animals infected with ICP10ΔPK (18 +/−1.3% at 20:1) was higher than that of unfractionated LNC from animals given HSV-2 (7 +/−0.4% at 20:1). Depletion of CD8+ CTL cells caused a significant reduction in the CTL activity of the LNC from ICP10ΔPK immunized mice (% lysis=46.7, 34.1 and 28.7 at 80:1, 40:1 and 20:1 relative to 100% for unfractionated cells). Depletion of the CD4+ cells did not decrease the CTL activity. By contrast, the CTL activity of LNC from HSV-2 infected animals was decreased by depletion of CD4+ cells (% lysis=43.7, 38.9 and 26.1 at 80:1, 40:1 and 20:1 relative to 100% for unfractionated cells). Depletion of CD8+ cells did not reduce the CTL activity. The data indicate that ICP10ΔPK induces CD8+ CTL which are not induced HSV-2.

Significantly, LNC from mice infected with ICP10ΔRR also had CTL activity (13 +/−0.6% at 20:1). The activity was significantly decreased by depletion of the CD4+ T cells, albeit to a somewhat lower level than that seen for HSV-2 (% lysis=154.7, 55.6 and 50.1 at 80:1, 40:1 and 20:1 relative to 100% for unfractionated cells). A minimal reduction in the CTL activity was also seen by depletion of CD8+ cells, but only at the high E:T ratios (% lysis=77, 74 and 89 at 80:1, 40:1 and 20:1 relative to 100% for unfractionated cells), suggesting that CTL activity is primarily mediated by CD4+ cells, as also seen for HSV-2. Without wishing to be bound by theory, the data can be interpreted to indicate that the ICP10 PK protein interferes with the induction of CD8+CTL.

### Example 4

ICP10ΔPK induces an HSV memory response similar to that of HSV-2

Having shown that ICP10ΔPK favors induction of local (LNC) and systemic (serum) Th1 responses including CD8+ CTL shortly after re-exposure to virus (second or third boost), we wanted to know whether it was capable of inducing a long term immune memory. To address this question a memory CTL assay was used.

The materials and methods used in the experiments presented in this Example are now described.

BALB/c mice were infected with ICP10ΔPK (3×10⁶ pfu) or HSV-2 (1×10⁶ pfu) by intraperitoneal inoculation and spleen cells were collected at 30 days post-infection. Cells (1×10⁷/well) were cultured in 24-well plates together with mKSA cells infected with HSV-2 (5×10⁵ pfu) for 16 hours and mitomycin C treated (50 μg/ml; 30 minutes). At 5 days in culture, the cells were collected and used as effectors in CTL assays done as described above. The results indicate that the memory CTL activity was similar for both viruses, indicating that ICP10ΔPK induces a memory CTL response similar to that of HSV-2.

Without wishing to be bound by theory, these studies indicate that relative to HSV-2, ICP10ΔPK favors induction of HSV-specific Th1 immunity including CD8+ CTL. This response is both local (LNC) and systemic (serum) and it appears to be mediated by increased IL-12 production by dendritic cells. Th1 including CD8+ CTL is a rapid response to re-exposure to viral antigen and can, therefore, contain the replication of the reactivated ganglionic virus thereby preventing the development of recurrent disease. By contrast, the immune response in animals infected with HSV-2 is skewed toward Th2 functions (likely related to lower IL-12 production by dendritic cells) and there are no detectable levels of CD8+ CTL. As such, the replication of the reactivated ganglionic virus proceeds unimpeded, resulting in development of recurrent disease.

### Example 5

HSV specific antibody in human patients with infrequent recurrences is primarily IgG1

The conclusion that patients with infrequent recurrent HSV have predominant virus-specific Th1 responses is supported by the subclass of virus specific immunoglobulin that is present in the serum. Sera were collected from 15 subjects with a history of infrequent HSV recurrent infections and assayed for virus specific antibody by ELISA. HSV-specific IgG, IgG1 and IgG2 were assayed, as described in Example 1. Briefly, ELISA plates were coated with HSV-2 antigen (50 μg/ml) or serial dilutions of human IgG (Sigma, St. Louis Mo.), IgG1 (Sigma) or IgG2 (Chemicon, Temicuba, Calif.) (100 μg/ml-2 ng/ml) used as control for standard curves, and incubated overnight at 4° C. The plates were washed with PBS-Tween and blocked with PBS-10% FCS for 1 hour at 37° C. The plates were washed and sera (diluted 1:5 and 1:50) were added to the HSV plates. After 2 hours at 37° C., the wells were washed and mouse anti-human IgG, IgG1 or IgG2 specific antibody conjugated to horseradish peroxidase (Southern Biotechnology Associates) was added. After incubation for 1 hour at 37° C. and subsequent washing, the substrate 2,2-azino-bis 3-ethylbenz-thiasoline-6-sulfonic acid (ABTS) was added to the wells for color development. The wells were read at 405 nm with an ELISA 2550 EIA reader (BioRad, Hercules, Calif.). Total levels of HSV-specific IgG were 9460 +/−3204 ng/ml. The levels of HSV-specific IgG1 were 7587 +/−to 3045 ng/ml and there was no obvious difference between HSV-2 and HSV-1 infected patients. The levels of IgG2 were 898 +/−512 ng/ml and there was no obvious difference between HSV-2 and HSV-1 infected patients.

Without intending to be bound to a particular mechanism of action, it is concluded that a method to treat HSV-2 recurrent disease involves activation of the cytokine cascade which: (i) begins with increased production of IL-12 by dendritic cells, (ii) is followed by modulation of the HSV-specific responses in favor of CD4+ Th1 responses and increased levels of IFN-γ (and decreased levels of IL-10), and (iii) in turn, results in increased levels of HSV-specific CD8+ CTL. This may be achieved by immunization with ICP10ΔPK, but will be equally effective in reducing recurrence if induced with other HSV recombinants, mixtures of virus proteins, nucleic acids, and polypeptides. The present invention is effective not only against HSV-2, but it is also effective against HSV-1. The present invention also discloses a method for identifying agents which induce a viral specific Th1 immune response and a method for the use of a combination vaccine that protects against latent and primary virus infection and induces a viral specific Th1 immune response.

Other methods which were used but not described herein are well known and within the competence of one of ordinary skill in the art of immunology, virology, cell biology and molecular biology.

The disclosures of each and every patent, patent application, and publication cited herein are hereby incorporated herein by reference in their entirety.

While this invention has been disclosed with reference to specific embodiments, it is apparent based on the disclosure provided herein that other embodiments and variations of this invention may be devised by others skilled in the art without departing from the true spirit and scope of the invention. The appended claims are intended to be construed to include all such embodiments and equivalent variations.

What is claimed is:

1. A method of ameliorating symptoms of a herpes simplex virus associated disease in an animal infected with a herpes simplex virus, said method comprising administering to said animal at least one immunogenic protein from said virus, wherein said protein induces a T Helper Cell type 1 (Th1) response.

2. The method of claim 1, wherein said Th1 response comprises one or more of the following responses:
a. an increased ratio of virus specific immunoglobulin subclasses reflective of a preferential Th1 response;
b. an increased virus specific interferon γ/interleukin-10 (IFNγ/IL-10) ratio;
c. increased CD8+ Cytotoxic T Lymphocyte (CTL) levels; and
d. increased Interleukin 12 (IL-12) levels.

3. The method of claim 2, wherein said increased ratio of virus specific immunoglobulin subclasses is selected from the group consisting of IgG2a/IgG1, IgG1/IgG4, IgG2/IgG4, IgG3/IgG4, (IgG1+IgG2+IgG3)/IgG4, (IgG1+IgG2+IgG3)/IgG5, IgG1/IgE, IgG2/IgE and IgG3/IgE.

4. The method of claim 1, wherein said at least one immunogenic protein when administered to a mouse induces a Th1 response comprising an increased ratio of IgG2a/IgG1.

5. The method of claim 2, wherein the Th1 response comprises an increased ratio of virus specific immunoglobulin subclasses reflective of a preferential Th1 response, an increased viral specific interferon γ/interleukin-10 (IFNγ/IL-10) ratio, increased CD8+ CTL levels, and increased IL-12 levels, by at least 25% each.

6. The method of claim 2, wherein said method results in an increase of the response comprising an increased ratio of virus specific immunoglobulin subclasses reflective of a preferential Th1 response by at least 25%.

7. The method of claim 2, wherein said method results in an increased viral specific interferon γ/interleukin-10 (IFNγ/IL-10) ratio, by at least 25%.

8. The method of claim 2, wherein said method results in increased CD8+ CTL levels, by at least 25%.

9. The method of claim 2, wherein said method results in increased IL-12 levels, by at least 25%.

10. The method of claim 2, wherein said animal is a human.

11. The method of claim 10, wherein said herpes simplex virus is a herpes simplex virus-2.

12. The method of claim 10, wherein said herpes simplex virus is a herpes simplex virus-1.

13. The method of claim 11, which comprises administering a composition comprising multiple herpes simplex virus-2 proteins in a pharmaceutically acceptable carrier, but not the ICP10PK protein.

14. The method of claim 10, which comprises administration of a virus which comprises the at least one immunogenic protein or which expresses the at least one immunogenic protein following administration.

15. The method of claim 14 which comprises administering a herpes simplex virus-2.

16. The method of claim 15 which comprises administering the ICP10ΔPK mutant of herpes simplex virus-2.

17. The method of claim 1, wherein the at least one immunogenic protein is administered indirectly by administering nucleic acids encoding the at least one immunogenic protein.

18. The method of claim 17, wherein said animal is a human.

19. The method of claim 18, wherein said viral disease is herpes and wherein said nucleic acid does not encode ICP10PK.

20. The method of claim 5, wherein said animal is a human.

21. The method of claim 20, wherein said herpes simplex virus is a herpes simplex virus-2.

22. The method of claim 20, wherein said herpes simplex virus is a herpes simplex virus-1.

23. The method of claim 20, which comprises administration of a virus which comprises the at least one immunogenic protein or which expresses the at least one immunogenic protein following administration.

24. The method of claim 21, which comprises administering a composition comprising multiple herpes simplex virus-2 proteins in a pharmaceutically acceptable carrier, but not the ICP10PK protein.

25. The method of claim 21, which comprises administering the ICP10ΔPK mutant of herpes simplex virus-2.

26. The method of claim 5, wherein the at least one immunogenic protein is administered indirectly by administering nucleic acids encoding the at least one immunogenic protein.

27. The method of claim 26, wherein said animal is a human.

28. The method of claim 27, wherein said viral disease is herpes and wherein said nucleic acid does not encode ICP10PK.

29. A therapeutic vaccine for ameliorating symptoms of a herpes simplex virus associated disease in an animal infected with a herpes simplex virus, said therapeutic vaccine comprising at least one immunogenic protein from the virus, which following administration to the animal induces a response comprising an increased ratio of virus specific immunoglobulin subclasses reflective of a preferential Th1 response, an increased viral specific interferon γ/interleukin-10 (IFNγ/IL-10) ratio, increased CD8+ CTL levels, and increased IL-12 levels.

30. The therapeutic vaccine of claim 29, wherein said herpes simplex virus is herpes simplex virus-2 and said at least one immunogenic protein from a herpes simplex virus is from a herpes simplex virus-2.

31. The therapeutic vaccine of claim 30, which comprises a herpes simplex virus-2 mutant.

32. The therapeutic vaccine of claim 31, wherein said mutant encodes a mutant ICP10 protein which lacks protein kinase activity.

33. The therapeutic vaccine of claim 29, said vaccine further comprising an immune stimulant or adjuvant.

34. The therapeutic vaccine of claim 29, wherein said herpes simplex virus is herpes simplex virus-1 and said at least one immunogenic protein from a herpes simplex virus is from a herpes simplex virus-1.

35. The therapeutic vaccine of claim 34, which comprises a herpes simplex virus-1 mutant.

36. A method of identifying an agent which ameliorates a herpes simplex virus associated disease in an animal infected with herpes simplex virus, said method comprising administering a test agent to an animal, analyzing the immune response thereto, and selecting a test agent that induces a Th1 response.

37. The method of claim 36, wherein said Thl response comprises an increased ratio of virus specific immunoglobulin subclasses reflective of a preferential Th1 response, an increased viral specific interferon γ/interleukin-10 (IFNγ/IL-10) ratio, increased CD8+ CTL levels, and increased IL-12 levels.

38. The method of claim 37, wherein said test agent is selected from the group consisting of a virus, a mutant virus, DNA, a polynucleotide, a protein, a peptide, and mixtures thereof.
